(19) United States
(12) Patent Application Publication
(10) Pub. No.: US 2024/0216211 A1
(43) Pub. Date: Jul. 4, 2024

(54) PRESSURE DIFFERENTIAL THERAPEUTIC ACTUATOR

(71) Applicant: Regents of the University of Minnesota, Minneapolis, MN (US)

(72) Inventors: Daniel M. Broberg, Deephaven, MN (US); Sergei A. Grishan, Moscow (RU); Linda A. Koehler, Scandia, MN (US); Essa A. Mohamed, Rochester, MN (US)

(21) Appl. No.: 18/563,628

(22) PCT Filed: May 25, 2022
(86) PCT No.: PCT/US2022/030893

§ 371 Date: Nov. 22, 2023

Related U.S. Application Data
(60) Provisional application No. 63/193,273, filed on May 26, 2021.

(51) Int. Cl. A61H 9/00

(52) U.S. Cl. CPC A61H 9/0007; A61H 9/0057; A61H 2201/1238; A61H 2201/1409; A61H 2201/165; A61H 2201/5002; A61H 2201/5056; A61H 2209/00

(57) ABSTRACT

An apparatus includes a membrane, a plurality of cup elements, and a pneumatic distribution port. The membrane has a conformable substrate and is configured for placement on a tissue site. The plurality of cup elements includes a first cup element and a second cup element. The first cup element and the second cup element are each disposed on a first side of the membrane, and each have a concave surface configured to engage tissue at the tissue site. The pneumatic distribution port has an inlet and an outlet. The outlet is coupled to the first cup element or is coupled to the first cup element and the second cup element.

### CLAIM OF PRIORITY

This patent application claims the benefit of priority of U.S. Provisional Patent Application Ser. No. 63/193,273, filed on May 26, 2021 (Docket No. 600.A83PRV), which is hereby incorporated by reference herein in its entirety.

### TECHNICAL FIELD

This document pertains generally, but not by way of limitation, to an actuator for providing therapy using pressure differential.

### BACKGROUND

The lymphatic system is part of the immune system. The lymphatic system carries fluid throughout the body, including waste, bacteria, and viruses. The lymph nodes filter out the waste which is eventually excreted from the body. Certain medical conditions and disorders can result in the fluid backing up in soft tissue, sometimes leading to damage of the lymph nodes. In some cases, the lymph vessels become obstructed.

Lynphedema is swelling due to the build-up of lymph fluid in the body. Lymphedema is frequently observed in the arms or legs of patients; however, it can occur in other parts of the body as well. Sometimes this swelling develops fairly quickly, or it may develop slowly over a course of several months. The exact mechanisms are not well understood.

Lymphedema may be related to various conditions. For example, cancer surgery sometimes entails removal of lymph nodes. Radiation therapy can damage nearby tissue that might include lymph nodes or lymph vessels. Infections can damage surrounding tissue (i.e., scarring). Other health conditions, such as obesity, heart failure, chronic venous insufficiency of low extremities can lead to lymphedema. Genetic alterations or mutations can involve the lymphatic system. Injury or trauma to a certain area of the body can lead to lymphedema.

In addition, health problems are often caused by lymphedema. For example, lymphedema increases the risk of having an infection in the swollen area. This is due to the cells that prevent infection and cannot reach a certain portion of the body. Wounds may heal relatively slower in the areas affected by lymphedema. Patients may feel upset, depressed, embarrassed, or angry about the physical disfigurement caused by lymphedema status. Joints in the part of the body with lymphedema may feel stiff or sore. Inflammation of the skin and connective tissues, known as cellulitis, can also be associated with lymphedema. Inflammation of the lymphatic vessels (lymphangitis) is also associated with lymphedema. Deep venous thrombosis (formation of blood clots in the deeper veins) is also associated with lymphedema. Cosmetic concerns are also associated with lymphedema. According to some research, there is a 10% chance of developing cancer of the lymphatic vessels known as lymphangiosarcoma for those patients with lymphedema.

Currently, there is not a cure available to patients with lymphedema. Treatments are designed to reduce swelling and control discomfort and other symptoms. Compression treatments can help reduce swelling and prevent scarring and other complications. Examples of compression treatments include elastic sleeves or stockings, bandages, manual drainage, and pneumatic compression devices.

Elastic sleeves or stockings must fit properly and provide gradual compression from the end of the extremity toward the trunk.

Bandages are wrapped more tightly around the end of the extremity and wrapped more loosely toward the trunk, to encourage lymph flow out of the extremity toward the center of the body.

Manual drainage includes massage techniques which can be useful for some people with lymphedema.

Pneumatic compression devices include sleeves or stockings connected to a pump that provides sequential compression from the distal end of the extremity toward the body. These may be used in the clinic or the home and are useful in preventing long-term scarring, however, they cannot be used in all individuals. For example, patients with congestive heart failure, deep venous thrombosis, or certain infections are not eligible to use such types of devices. Surgical treatments for lymphedema are used to remove excess fluid and tissue in severe cases, but no surgical treatment is able to cure lymphedema.

### SUMMARY

One example of the present subject matter provides alternating negative and positive pressure in a wearable device to restore lymph and blood movement (circulation) and tissue composition.

One example of the present subject matter provides treatment for lymphedema and phlebolymhedema. An example of the present subject matter can be used to treat superficial lymphatic system malfunction which might be characterized by swelling, cellulite, cellulitis, or other possible clinical manifestations.

One example includes a wearable device configured to apply modulated differential pressure (such as negative and positive pressure) to the skin surface of the human body for external stimulation of lymphatic and blood circulation in the underlying subcutaneous tissue and the skin itself. The modulated pressures, which can include alternating negative and positive pressure, can allow improvement in tissue composition, particularly prevention and alleviation of fibrosis development through controllable local stretching of collagen fibers in underlying tissues.

One example device includes an actuator in the form of a wearable patch configured to be coupled at a tissue site. The actuator can include a pliable base having a plurality of hollow chambers (or cups) which penetrate the base and are attached to it. One surface of the base is covered with an adhesive substance for providing contact with the skin. Hollow chambers are arranged on the base in a pattern configured for exerting forces on the tissue. In one example, the hollow chambers are connected by tubes to a pump. The pump is configured to provide negative pressure (that is, below ambient) or positive pressure (that is, above ambient). The pump, in one example, includes a pressure controller for adjustment of pressure parameters, duration, and selection of a modulation regime.

According to one example, a pliable base has an adhesive surface. The base is configured to carry a plurality of hollow chambers interconnected through the tubing to the pump and pump controller. In one example, the pliable base includes an elastic material (polymer) with sticky layer on one side (underside) for contact with the skin. The arrangement of hollow chambers can be configured in a pattern or otherwise distributed on the surface. The hollow chambers are affixed to the base in a pattern for even distribution of negative or positive pressure on the skin surface.

In one example, an adhesive bonds an actuator to tissue. An adhesive bond can be configured to withstand the atmospheric pressure applied within the cup. According to one example, a low negative pressure helps to keep the cup in place on the tissue.

In one example, the actuator is fabricated of foam sheet stock having an adhesive surface.

A plurality of cups can be arranged in an ordered pattern of rows or in a scattered distribution. One example includes a distribution of cups in a manner similar to a honeycomb.

According to one example, the particular arrangements of tubes are configured to connect the hollow cups to one or more pumps for even and simultaneous distribution of negative or positive pressure. Regimes of negative and positive pressure cycles can be tailored for various pathological conditions and individual anatomical characteristics of the patient.

According to one example, the present subject matter can be deployed for treatment of lymphedema, soft tissue fibrosis, disturbed blood circulation, chronic venous insufficiency, cellulite, and for rehabilitation.

Examples of the present subject matter can be used to solve medical problems. For example, one configuration can address disturbed lymph circulation. As another example, one configuration can address disturbed blood circulation. As another example, one configuration can address soft tissue fibrosis.

One example of the present subject matter is wearable and provides treatment using a pressure differential. The pressure differential can include any number of pressures at pressure values in the range of −200 mm Hg to 200 mm Hg. One example operates with pressure values in the range of −50 mm Hg to 50 mm Hg.

That is, a first pressure can be negative, and a second pressure can be positive. One example is wearable on patient and is adjustable for various sizes and individual anatomical characteristics of the wearer. The pressure, frequency, and duration can be individually adjusted via a pump controller algorithm.

Examples of the present subject matter can be used in a residential setting or at a clinical setting and configured for treatment of lymphedema, chronic venous insufficiency, soft tissue swelling, disturbed soft tissue elasticity and fibrosis development, restoration of lymph and blood circulation locally in cases of increased physical activity and for restoration thereafter.

Each of these non-limiting examples can stand on its own or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates a partial section view of a cup, according to one example.

FIG. 2A illustrates a system schematic, according to one example.

FIG. 2B illustrates a system schematic, according to one example.

FIG. 3A illustrates a view of an actuator, according to one example.

FIG. 3B illustrates a partial section view of an actuator, according to one example.

FIG. 4A illustrates a view of a cup, according to one example.

FIG. 4B illustrates a partial section view of a cup, according to one example.

FIG. 5 illustrates an assembly, according to one example.

FIG. 6 illustrates a view of a plurality of cups, according to one example.

FIG. 7 illustrates pneumatic lines and an assembly, according to one example.

FIG. 8 illustrates an element of an assembly, according to one example.

### DETAILED DESCRIPTION

Treatment of lymphedema can take several forms. One approach includes opening and recanalizing obstructed lymphatic vessels (particularly in the proximal regions relative to the affected area of the body). Another approach entails improving the motility of the lymphatic vessels and pressing out the lymph into the centripetal direction (from the distal parts of the body to the proximal ones). Another approach entails collateralization of lymphatic circulation.

Negative pressure, in the range of −20 to −200 mm Hg, when applied superficially on the skin, opens the subcutaneous lymphatic capillaries and conductive vessels, and can reduce or eliminate existent obstructions blocking the normal lymphatic flow.

Positive pressure, in the range of +20 to +200 mm Hg, when applied superficially on the skin, can stimulate the propulsion of the lymphatic vessels, and press the lymph out of them.

According to one embodiment, an example of the present subject matter includes a base made of the pliable material, which can be adjusted to various body contours.

The base can have various shapes and sizes. In one example, the base has a shape of a rectangular sheet or patch. A first side of the base is covered with a layer of adhesive material. The adhesive forms a contact or seal between the skin of the body and the base.

In one example, an adhesive includes medical-grade silicone adhesive gel such as that mentioned in https://iopscience.iop.org/article/10.1088/2057-1976/aa91fb.

The actuator can be bonded to tissue with contact sufficient to withstand the pressure range from +200 mm Hg to −200 mm Hg without excessive air leakage. The assembly can be bonded to tissue with an adhesive. Hair, scar tissue, or other irregularity in the tissue site may lead to some leakage. The pump and pressure monitoring system can be configured to tolerate a small leak. In one example, the contact between the cups and the tissue is a sealed surface and in one example, the interface is impermeable for air.

The actuator can be attached or coupled to the tissue site by various features.

In one example, the actuator is attached to the tissue site using a medical grade adhesive. For example, a foam-based assembly can be configured with a laminate and adhesive having a release liner. The adhesive can tolerate small leakage provided the pumping system can overcome the loss of the leak. With a vacuum system, the adhesive need not connect to the skin as the vacuum alone can retain the assembly on the tissue site. When pressurized, the cups are held securely to the tissue. In one example, the actuator includes a cuff held with hook and loop (Velcro). In one example, the actuator is held in place with a compressive-type garment made of spandex. In one example, the actuator is retained on the tissue site using a fastening mechanism that allows for unrestricted flow. In one example, a garment of neoprene foam has integrated, or built-in, chambers.

Attached to the base is a plurality of cups or chambers. Each chamber penetrates the base through the opening of the corresponding size and shape.

In one example, chambers are rectangular in shape, placed in sequential order, adjacent to each other with spaces between them. In one embodiment, chambers are organized in parallel rows. The chamber is hollow inside, made of plastic or any other stiff material not permeable to the air.

On a contact side of the base, the chamber is open. Through this opening, the chamber contacts the tissue and through this opening, negative or positive pressure is applied to the tissue. In one example, each chamber is fluidly connected to the hollow tube.

One example of the present subject matter can be fabricated using a commercial foam fabricator service and includes laminating various sheet stock materials together. Low-cost tooling, such as steel rule dies, can be used to cut foam. In one example, the foam is formed to have channels that communicate to each cup. In one example, tubing is set in the foam and contains hypo tubing that be positioned to penetrate the foam and access the cup with the pump sized and configured to overcome any minor leak.

In one example, the chambers or cups are configured to penetrate the base. As such, the open end of the cup has unhindered contact with the tissue surface. In one example, a screen or porous film is in contact with the skin. In one example, the actuator can be operated in a manner to simulate a manual massage including stretching or pulling on the skin by means of an applied vacuum or pressure. The tissue is manipulated, or massaged, by pulling or drawing into the cup. A screen can be configured or sized to allow application of vacuum and allow for the tissue to stretch.

The tubes from each chamber are connected to the pump. The tubes can be connected to the pump in a one-to-one relation or can be connected in a many-to-one relation in tubes from more than one chamber are combined in a cluster or a group, and as a whole, the group is coupled to the pump.

In one embodiment, a pump is a detached unit, that can be coupled to a belt (such as a waist belt) and worn on the patient's body.

The pump includes an electric motor having a shaft coupled to a device that moves a gas in this embodiment. A pump controller can include an electronic computational device that controls the pump and sets up the pressure in chambers. In this example, the controller is located in the same housing as the pump.

In one example, a cup has dimensions in the range of 1 cm to 10 centimeters either in diameter or if square along one of the edges. The cup dimensions can be larger than 10 cm or smaller than 1 cm.

In one example, the depth of the cup allows the tissue to be gently stretched. In one example, the depth of the cup can be 1 centimeter for small sizes and 2 to 3 centimeters for larger sizes. The cup lower limit dimension is sized to be effective over a very small surface area and at the upper limit, sized as a function of anatomy for bonding with a consideration being the ability of the cup to seal around the perimeter.

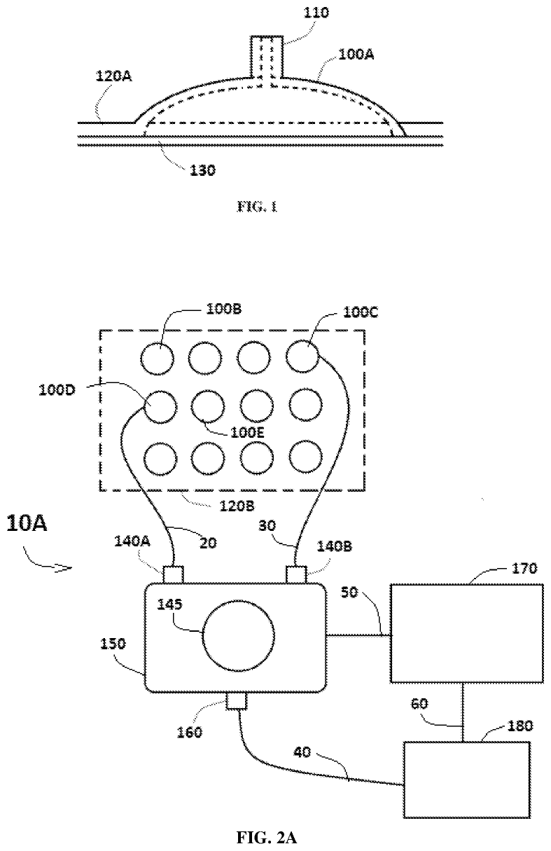

FIG. 1 illustrates a partial section view of cup 100A, according to one example. The cup, in some examples, can be described as a chamber. In this view, the upper side of cup 100A includes port 110. Port 110 is fluidly coupled to the interior of cup 100A. The underside of cup 100A includes a void. Cup 100A is affixed to membrane 120A. Membrane 120A, as shown here, has adhesive 130 on an underside. Adhesive 130 is configured to bond to tissue of a patient.

Cup 100A has sufficient rigidity to substantially avoid collapse when subjected to a negative pressure within the void and sufficient rigidity to avoid substantial distortion when subjected to a positive pressure within the void. Suitable materials for cup 100A are described elsewhere.

In the example shown, port 110 is affixed to cup 100A by bonding or formed integrally with cup 100A. Port 110 can include a threaded or threadless fluid coupling. In one example, port 110 includes a partial-turn coupling mechanism, such as a Luer-lock coupling.

Membrane 120A can include a pliable laminated structure. Membrane 120A can be configured to conform with a tissue site on a body. Membrane 120A can include a sheet-like structure.

Membrane 120A can include a closed cell foam. The foam can include a polymer material. The cups affixed to membrane 120A can be injection molded of a thermoplastic elastomer or a more rigid thermoplastic in combination with a thermoplastic elastomer (an example is the soft grip over-molded on a toothbrush). In various examples membrane 120A includes silicone or a neoprene foam. Membrane 120A can be fabricated by sewing.

Membrane 120A can be described as pliable or elastic (polymer). A pliable, non-toxic material suitable for medical applications (polymer, rubber, silicone, etc.). A durometer range can be selected. In one example, the material is nonporous and allows for the easy transmission of air. In one example, membrane 120A is nonelastic and is sufficiently flexible to comply or conform with tissue at the body portion for treatment.

Cup 100A can have a wall of elastic or inelastic material. For example, a wall of cup 100A can be fabricated of glass or a hard plastic such as polycarbonate or acrylic.

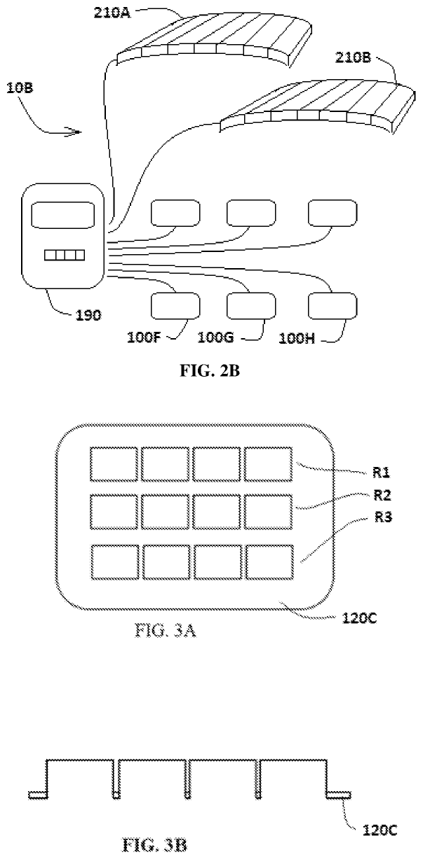

FIG. 2A illustrates schematic of system 10A, according to one example. The illustrated system includes a membrane 120B having a plurality of cups (some of which are marked as cups 100B, 100C, 100D, and 100E), distributor 150, valve 145, controller 170, and pump 180.

Membrane 120B in this example includes twelve cups arranged in a matrix of rows and columns. Cup 100C and cup 100D of the matrix are each shown to be separately coupled to ports 140A and 140B of distributor 150, and other cups are also connected to ports as well. The cups in this example are each separately coupled by lines to a corresponding port on the distributor. The lines in this example are fluid lines configured to remain flexible and carry air or other gaseous fluid.

Distributor 150 includes valve 145. Distributor 150 can include a plurality of channels or passageways through which pneumatic pressure is conveyed. In one example, distributor 150 includes a plurality of discrete lines or channels arranged in point-to-point configuration. Distributor 150 can include a plurality of channels or passageways through which pneumatic pressure is conveyed. In one example, distributor 150 includes a plurality of lines or channels formed in a unitary manner by molding, photolithography, additive manufacturing, or other process.

Valve 145 is coupled to distributor 150 in a manner to fluidly make and break passageways between inlet port 160 (shown here at the bottom of distributor 150) and selected ones of the outlet ports 140A and 140B (shown here at the top of distributor 150). Valve 145 can be a gate valve, a rotary valve, a reed valve, a shuttle valve, or other type of valve.

Inlet port 160 of distributor 150 is coupled to pump 180 by fluid line 40. Fluid line 40 can be a multi-channel line that conveys one or more fluid pressures between pump 180 and distributor 150. For example, a first channel of line 40 (between pump 180 and distributor 150) can carry a gas at a first pneumatic pressure and a second channel of line 40 can carry a gas at a second pneumatic pressure. The first pressure and the second pressure can be different. The channels of line 40 can carry differential pressures simultaneously. In one example, the line between the pump and the distributor includes a single channel and the different pressures provided by pump 180 are applied at differing times.

Pump 180, in various examples, can have a variety of configurations. For example, pump 180 can include a diaphragm, a peristaltic element, a scroll element, a piston, or other structure configured to provide pressure. In other examples, a first pump provides a first pressure, and a second pump provides a second pressure, and the plurality of pumps are each coupled to a respective inlet port 160 of distributor 150.

In one example, different pressures are applied to different cups in a managed time-ordered sequence. Additionally, the pressures in the cups are changing in relationship to each other. The pressures are not static, but dynamic and also dynamic relative to other cups. In one example, the modulated pressures within the cups of the present subject matter simulates the action of a manual massage in terms of stretching tissue followed by relaxation of the tissue. Pressure can be modulated between a positive value (that is, above an atmospheric pressure) and a negative value (that is, below an atmospheric pressure). Pressure can also be modulated between different positive values or modulated between different negative values. In one example, one pressure is at an ambient (or atmospheric) pressure and a second pressure is either a positive or negative value.

Controller 170 in system 10A is coupled by signal line 50 with distributor 150 (and valve 145) and coupled by signal line 60 with pump 180. Signal lines 50 and 60 can include electrical conductors or optical lines. Controller 170 can include a processor configured to control operation of pump 180, valve 145, and distributor 150. In one example, controller 170 is wirelessly coupled. Controller 170 can include an analog processor or a digital processor with suitable instructions or programming to control and operate the pressure conveyed to the cups.

FIG. 2B illustrates a schematic of system 10B, according to one example. In this example, module 190 includes a pump, a distributor, a valve, and a controller. Module 190 is shown coupled to a plurality of discrete ‘single’ chambers, some of which are marked as cups 100F, 100G, and 100H in one-to-one relation. A ‘single’ chamber includes a cup and membrane assembly, and in the example shown, each is coupled to module 190 by a discrete line.

Module 190 is also coupled to multi-chambered patches 210A and 210B, each of which can be viewed as an actuator. Patches 210A and 210B shown here include a plurality of linear chambers with each linear chamber having a plurality of cups. Patches 210A and 210B are each coupled to module 190 by a multi-channel line.

Illustrated module 190 includes a visual display and user-operable controls by which a user can monitor the system and provide input to control the operation.

FIG. 3A illustrates a view of an actuator, according to one example. The illustrated actuator includes a membrane 120C and a plurality of cups arranged in rows R1, R2, and R3, and columns. The cups in this example are rectangular shape however a circular shape is also contemplated. The actuator illustrated can be interpreted as a view from a contact side, in which an adhesive bonds membrane 120C to the adjacent tissue and the void of each cup is visible. The actuator illustrated can also be interpreted as view from a non-contact side, in which case the upper surface of membrane 120C and the outer surfaces of each cup are visible.

FIG. 3B illustrates a partial section view of an actuator, according to one example. This view illustrates the coupling between membrane 120C and four cups. The cups are discretely selectable elements.

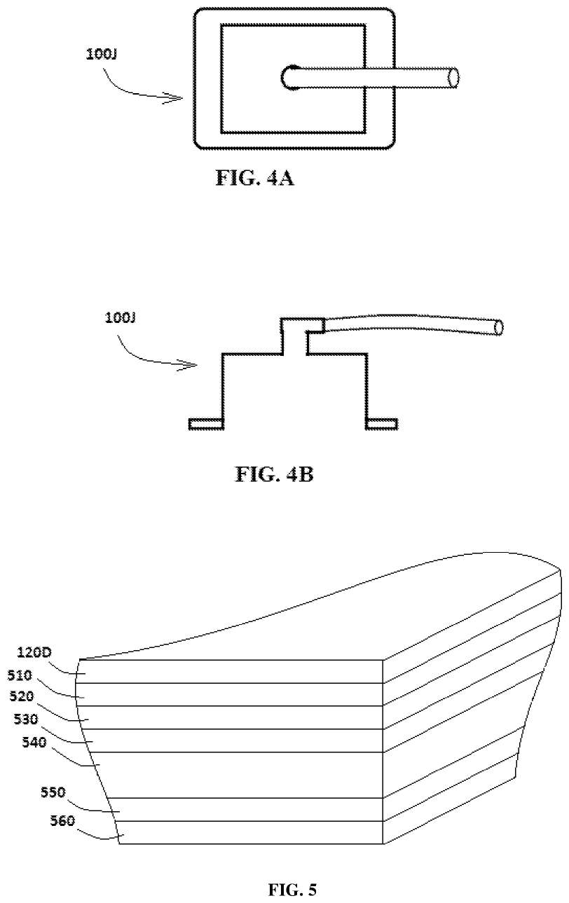

FIG. 4A illustrates a view of cup 100J having a line segment attached thereto, according to one example. The figure illustrates a view from above showing an outer surface of cup 100J and the membrane about the perimeter of the cup. The illustrated cup has a rectangular configuration, and the line is coupled in a manner without using a boss. In one example, the line is coupled to the walls of the bore in the cup or bonded on a surface of the void-side of the cup.

FIG. 4B illustrates a partial section view of cup 120J, according to one example. In this figure, the line is shown fluidly coupled to cup 120J.

FIG. 5 illustrates an actuator assembly, according to one example. The layers of the actuator assembly are visible in this view. In the example shown, the assembly includes membrane 120D, adhesive 510, closed cell foam 520, adhesive 530, closed cell foam 540, medical adhesive 550, and release liner 560. Membrane 120D (or film) is disposed on an upper surface. In the illustrated view, release liner 560 is at the bottom and provides protection for medical adhesive 550 at a time before placement on a tissue site. Release liner 560 is removable without using tools and when removed, medical adhesive 550 is exposed, and the assembly can be applied to the tissue site. Medical adhesive 550 is bonded to a first layer of closed cell foam 540. The first layer of closed cell foam 540 is bonded to other layers by adhesive 530.

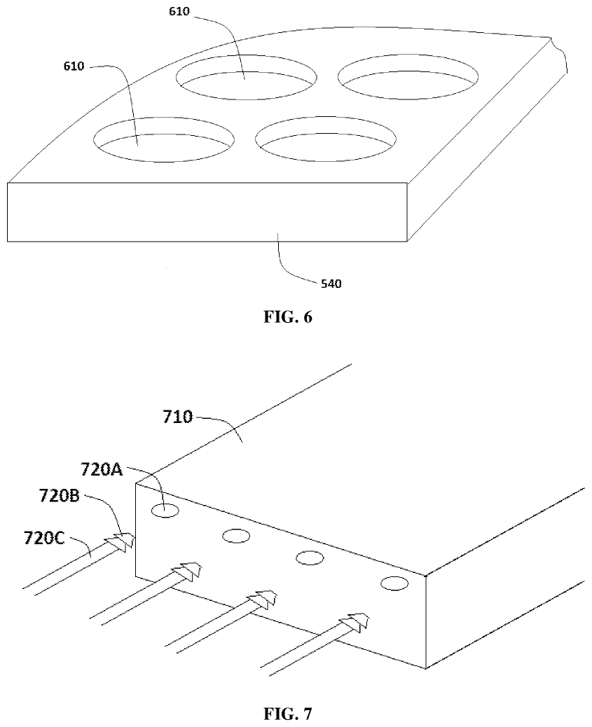

FIG. 6 illustrates a view of a plurality of cups, according to one example. In the example shown, closed cell foam 540 is configured to create the cups. The foam can be die cut with through holes 610.

FIG. 7 illustrates pneumatic lines (one of which is denoted here as line 720C) and an assembly, according to one example. The actuator assembly 710, sometimes called a patch, includes a plurality of channels, and in the figure, four channel openings are visible. One such channel opening is denoted here as opening 720A. The lines of the tubing set are terminated with hose barbs, one of which is denoted here as barb 720B, that engage with the channel openings. The hose barbs are flexible and conform in a manner to form fluid-tight couplings between the tubing and the channel openings. In the example shown, the four lines are discrete and individually coupled to the channel openings in one-to-one relation. In one example, a unitary plug provides coupling for a plurality of channel openings with a corresponding plurality of channels of a line set.

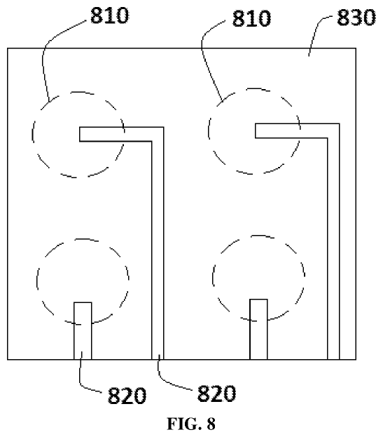

FIG. 8 illustrates an element of an actuator assembly, according to one example. In the figure, passageways 820 are shown to be cut from a second piece of foam 830. The passageways 820 are cut though the foam but not the membrane laminated to the foam. The material removed to form the passageways can be stripped out before the foam is laminated to a thicker block with large pockets. The assembly can be fabricated using tooling or dies that can cut through one layer without cutting through a second layer laminated to the first layer. For example, an adhesive can be laminated to the stock. An additional layer provides a release liner that covers the adhesive until the membrane is to be applied. The material can be kiss cut so that the die cuts through one lamination and the adhesive and not through the release liner. After cutting, the user can peel the membrane and attached adhesive, and the release liner stays intact. The passageways 820 are coupled to the replaced openings in the foam, here some of which are denoted openings 810.

### Various Notes

In one example, an assembly includes a single cup. The assembly can be coupled to a compressed air source and to a vacuum source (pumps). The source of pressure or vacuum can exceed the requirement for pressure or vacuum needs at the cup.

In one example a distribution manifold is provided. The assembly can be connected by a first chamber or channel to the pressure supply and connected to a second, separate, chamber or channel to the vacuum supply. A third chamber or channel independent from the first two can communicate to the tubing that connects to the cup.

In one example, the first chamber or channel is connected to a vacuum supply at one set point and connected to a second, separate, chamber or channel to a vacuum supply at a different set point.

In one example, a first valve connects the pressure chamber to the third chamber. A second valve is configured to connect the vacuum chamber to the third chamber. The valves are simple valves having either open or closed position. Flow is not regulated by the valve (it is not a proportional flow control valve). If pressure is desired the first valve is opened, applying pressure to the third chamber, and the second valve remains closed. The opposite configuration occurs to apply vacuum to the third chamber.

A pressure monitor is configured to measure the pressure (or vacuum) in the third chamber.

In one example, the system is controlled by software that executes an algorithm to determine both the application of pressure (or vacuum) and the duration.

When the algorithm calls for vacuum in the third chamber, the second valve is opened. The pressure sensor is configured to monitor the actual value and compare it to the programmed value. When the desired vacuum level is reached, the second valve will close. If there is a vacuum leak and additional vacuum is needed, then the sensor will signal a change, and the software then directs the second valve to pulse open. By pulsing the valve on and off, the pressure or vacuum can be regulated to a desired set point.

By pulsing the valve on and off the pressure or vacuum can be regulated to a fixed or changing set point. The pressure within the cup can be modulated up and down.

A tubing line is coupled to communicate from the third chamber in the manifold to the cup within the patch.

The tubing can be part of the patch, an independent tubing set, or part of the manifold.

Tubing can connect to the manifold via quick-connect fitting, hose barbs, or other tubing connectors. Multiple tubes can be connected to the manifold using a fitting that contains all the tubing connections for a patch. For example, a ten-tube connector can have a circular arrangement of tubing ports.

In one embodiment, each tube is connected by a hose barb. The hose barb can be inserted into the patch associated with each cup.

In one example, a multichannel connector mates with the actuator in a preplanned orientation so all the channels communicate with individual cups. This option could be held in communication with the patch with hook and loop material.

In one example, the actuator, or patch, is an open chamber and the tube is connected to a “lid” that snaps onto the top of the chamber like a lid on a food container.

The actuator can be coupled to a pump configured to provide both pressure and vacuum, pressure only, or vacuum only. The pump can be manual, or electric.

An electric version can be configured to operate using battery power. In one example, a double-diaphragm type pump is provided. An example uses one pump for pressure and a second pump for vacuum. In one example, a pump includes a manually-operated pump having a piston. In one example, a manual pump provides pressurized air, and a controller applies the proper pressure.

A transition from pressure and vacuum can include diaphragms and two chambers and the control software can pulse the valves to change the state in a selected cup.

In one example, a pump controller is located apart from the pump. The control software can be implemented in a computer or programmable logic controller (PLC). In one example, the pumps, controller, and a manifold are bundled into one package.

In one example, the actuator is configured to operate with a cycle duration of several seconds or several minutes.

In one example, a system of valves can be controlled to open and close quickly. For example, one mode enables following a curve of pressure verses time. An algorithm can include the following:
a) Open valve for fixed amount of time. Examples include a duration of one second or 50 milliseconds.
b) time delay for a fixed duration to allow the system to stabilize. In one example, pressure is sensed continuously and monitor to detect a minimum amount of variation.
c) Compare pressure to set point.
d) Determine if more pressure is needed.
e) Pulse valve open again and repeat a-d until the set point is reached.

This configuration allows walking up or down a curve or allows for oscillating between pressure and vacuum.

In one example, a pattern of pressure is configured to be intermittent and adjustable individually. The pressure range can be from −200 to +200 mmHg. Timing can be selectable, and in one example, configured for shifting between high-frequency oscillations (a few seconds interval) to low-frequency (a few minutes interval).

In one example, a system includes aspects to reduce heating at the tissue site. This can include sizing the assembly to reduce area of coverage. In one example, the actuator includes a garment. In one example, positive pressure in the cups is provided through the pressurized air current; the air current should be not higher in temperature than the ambient atmosphere, and thus provide cooling under the patch.

In one example, a Peltier effect cooling system is provided by which electric current through a junction between conductors can remove heat. A Peltier effect cooler can chill the air on the pressure side which may reduce swelling. A manifold having aluminum can be configured with a Peltier effect cooling system.

One example of the present subject matter includes an actuator having a plurality of channels and a plurality of cups. The channels and cups can be arranged to facilitate dynamic pressure modulation applied to the tissue site.

In some examples, the plurality of cups are separately and independently repositionable about a tissue site.

In some examples, the pressure applied at each cup, or to a selected subset of cups, is separately and independently controllable.

In one example, a pressure applied to a set of cups is modulated to form a gradient to address a particular medical condition. The modulation can be configured by suitable programming of a controller. For example, the cup pressures can be modulated in a controlled manner to massage a lymph system, a region of the lymph system, or other specific site. In another example, the pressure can be modulated to control or stretch tissue fibers (subcutaneously). In one example the actuator can be manipulated to control direction of lymph flow from distal to proximal regions of the body. If untreated, lymph fluid flow can be impeded by medical condition and this affects flow to the heart which can lead to collected fluid in the arms and other tissue, resulting in swelling.

One example of the present subject matter is configured to automate tissue massage in order to improve lymph flow. This includes stretching and relaxing the tissue in a sequence and modulation in a planned event. The sequence and modulation may be different for patients at various disease states.

A modulated pressure gradient can provide direct and mild massage of proximal lymph system as well as direct distention of elastic fibers and prevention of fibrosis.

In some examples, the actuator is coupled to a wearable garment or a wearable device. A wearable garment can include a sleeve-like device suitable for use on a leg or arm. In one example, the garment is applied to a trunk area of the patient. In one example, multiple discrete actuators are deployed on a patient in a manner to move from fluid in a direction from a limb to the trunk.

In some examples, the actuator is configured as a wearable device that allows the patient to independently position and operate in order to achieve a desired massage.

In some examples, the actuator can be pressure modulated to simulate a wave or modulated to move in a linear or controlled direction. For example, in a capillary bed, applied positive and negative pressures can open pores, allowing fluid to move into lymph ducts, then able to uptake fluids and move fluid from a distal site to a proximal site (centripetal).

In one example, the actuator has a rectangular planform and has typical dimensions of 5 cm by 10 cm. Other planforms and sizes are also contemplated.

In one example a single valve modulates pressure applied at an actuator based on delivered pressure from one, two, or more pumps.

In one example, a sensor is affixed to the actuator membrane. A sensor can provide an electrical signal corresponding to bioimpedance indicative of fluid within the tissue. A plurality of electrical nodes can be monitored for impedance and provide a signal to the controller for use in selecting a gas pressure applied to the actuator in a feedback loop.

In one example, the actuator provides physical manipulation of tissue in the manner of a manual massage. That is, stretching, compressing, and pulling of tissue to cause pressure changes to expand the tissue and open the lymph tissue. This can include dynamic manipulation in which the tissue is stretched and then allowed to contract, and then stretched and contracted again in a modulating fashion.

In one example, an applied vacuum expands and stretches the tissue in a perpendicular direction relative to the membrane.

In one example, differential pressures can be applied simultaneously or applied in a timewise modulation to form a wave-like modulation profile.

One example includes an apparatus having a membrane, a plurality of cup elements, a pneumatic distribution port, a distributor, and a controller. The membrane has a conformable substrate and is configured for placement on a tissue site. The plurality of cup elements includes a first cup element and a second cup element. The first cup element and the second cup element are each disposed on a first side of the membrane and each have a concave surface configured to engage tissue at the tissue site. The pneumatic distribution port has an inlet and an outlet. The outlet is coupled to the first cup element or coupled to the first cup element and the second cup element. The distributor has a valve, a pump port, a first port, and a second port. The pump port is configured to couple with a pump discharge port. The first port is fluidly coupled to the first cup element, and the second port is fluidly coupled to the second cup element. The valve is configured to selectively couple the pump port to the first port and to the second port. The controller is coupled to the distributor. The controller is configured to control operation of the valve whereby a first fluid pressure at the first port differs from a second fluid pressure at the second port. The controller is configured to selectively modulate the first fluid pressure and the second fluid pressure.

In one example, the controller is configured to modulate the first fluid pressure independent of the second fluid pressure.

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

The claimed invention is:

1. An apparatus comprising:
a membrane having a conformable substrate and configured for placement on a tissue site;
a plurality of cup elements including a first cup element and a second cup element, the first cup element and the second cup element each disposed on a first side of the membrane and each having a concave surface configured to engage tissue at the tissue site; and
a pneumatic distribution port having an inlet and an outlet, the outlet coupled to the first cup element or coupled to the first cup element and the second cup element.

2. The apparatus of claim 1 wherein the membrane comprises a garment.

3. The apparatus of claim 1 wherein the membrane comprises a fabric.

4. The apparatus of claim 1 wherein the membrane is configured for adhesive bonding to the tissue.

5. The apparatus of claim 1 wherein the first cup element is adjacent the second cup element.

6. The apparatus of claim 1 wherein the pneumatic distribution port includes a tubing network.

7. The apparatus of claim 1 wherein the pneumatic distribution port is configured to convey a gas.

8. The apparatus of claim 1 wherein the pneumatic distribution port is configured to convey fluid at a fluid pressure below atmospheric pressure.

9. The apparatus of claim 1 wherein the pneumatic distribution port is configured to convey a first fluid pressure to the first cup element and convey a second fluid pressure to the second cup element, wherein the first fluid pressure differs from the second fluid pressure.

10. An apparatus comprising:
a plurality of cup elements including a first cup element and a second cup element, the first cup element and the second cup element each having a concave surface configured to engage tissue at a tissue site; and
a pneumatic distributor having a first port coupled to the first cup element and having a second port coupled to the second cup element wherein a fluid pressure in the first cup element differs from the fluid pressure in the second cup element.

11. The apparatus of claim 10 wherein the pneumatic distributor is configured to modulate the fluid pressure applied to the first cup element independent of the fluid pressure applied to the second cup element.

12. The apparatus of claim 11 wherein the fluid pressure is modulated with respect to time.
